(19) United States
(12) Patent Application Publication
(10) Pub. No.: US 2004/0242981 A1
(43) Pub. Date: Dec. 2, 2004

(54) Method and apparatus for increasing aircraft safety

(76) Inventor: Ram Pattisapu, Irving, TX (US)

Correspondence Address: F. Lindsey Scott, Suite B, 2329 Coit Road, Plano, TX, 75075, US

(21) Appl. No.: 10/847,716

(22) Filed: May 18, 2004

Related U.S. Application Data
(60) Provisional application No. 60/474,463, filed on May 30, 2003.

(51) Int. Cl. A61B 5/00

(52) U.S. Cl. 600/323

(57) ABSTRACT

A method and apparatus for increasing aircraft safety by monitoring and providing a signal indicative of a blood oxygen level below a selected minimum of an aircraft pilot while in flight.

### FIELD OF THE INVENTION

This invention relates to a method and apparatus for increasing aircraft safety by monitoring and providing a signal indicative of the blood oxygen level of an aircraft pilot while in flight.

### BACKGROUND OF THE INVENTION

Low blood oxygen level (hypoxia) of a pilot is an insidious threat during high altitude operations in aircraft, whether or not pressurized. Low blood oxygen levels are a threat because pilots do not normally recognize many of the subtler effects of slightly lowered levels of oxygen in their blood stream, such as headaches, dullness, loss of depth perception, sleepiness, etc. In more severe cases, a loss of coordination, inability to access important information and the like could lead to catastrophic consequences for the aircraft. Further, in most cases the onset of the low blood, oxygen level will be gradual.

While many aircraft are routinely equipped with safety devices, such as autopilot systems, auto-land systems, emergency oxygen supply systems, altitude limiting systems and pressurization systems, these systems do not function in the absence of a suitable activation signal. Such systems are considered to be well known and are included in many aircraft, especially in larger aircraft. However, in the absence of pilot activation, many of these systems do not self-activate and thus are ineffective. There are problems associated with the activation of these systems automatically. Without the ability of the pilot to activate, shut down, or reset the systems, there are many instances where the systems remain inoperative even when the pilot may be suffering from low blood oxygen levels, which may cause the pilot to become increasing incapable of functioning.

According to the present invention, a system and method have been developed which are effective to alert the pilot to low blood oxygen levels by suitable indicators. The signal generated can also be used to activate systems for the continued safe operation of the aircraft.

### SUMMARY OF THE INVENTION

According to the present invention, it has been found that a system for increasing aircraft safety by monitoring and providing a signal indicative of a pilot's blood oxygen level comprises a blood oxygen level sensor in contact with a selected area of the pilot's body and, a signal generator in communication with the blood oxygen level sensor and adapted to generate a signal when the pilot's blood oxygen level drops below a selected blood oxygen level.

The present invention further comprises a method for increasing aircraft safety by monitoring and providing a signal indicative of the blood oxygen level of a pilot in an aircraft, the method comprising monitoring the blood oxygen level of the pilot and generating a signal indicative of the pilot's blood oxygen level when the pilot's blood oxygen level drops below a selected blood oxygen level.

### BRIEF DESCRIPTION OF THE DRAWINGS

FIG. 1 is a schematic diagram of an embodiment of the present invention; and,

FIG. 2 shows an alternate embodiment of the present invention.

### DESCRIPTION OF THE PREFERRED EMBODIMENTS

In the description of the Figures, the same numbers will be used throughout to refer to the same or similar components.

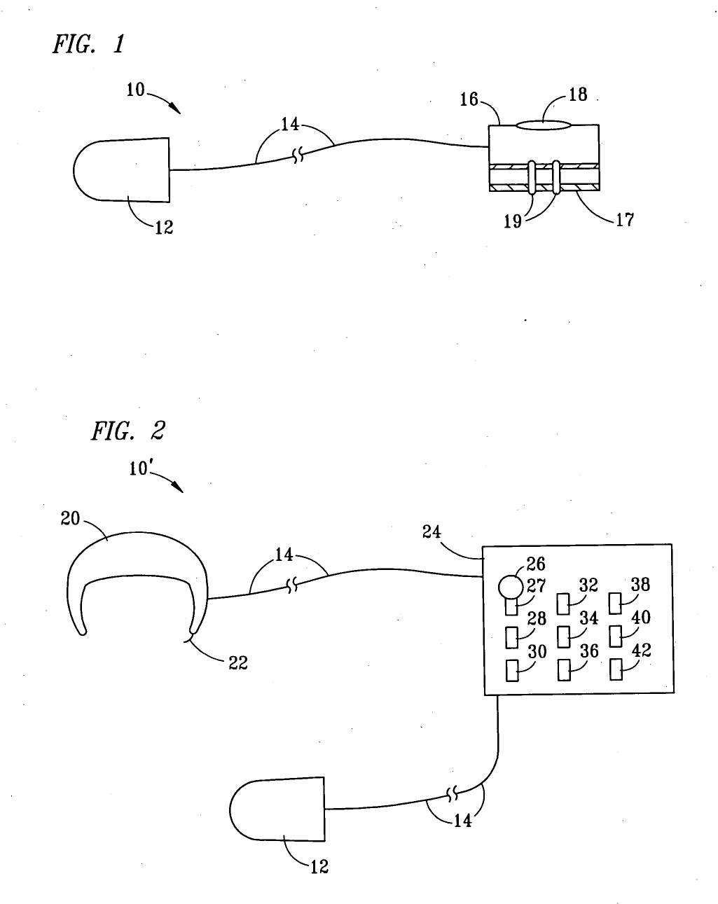

In FIG. 1, a relatively simple embodiment of the system of the present invention is shown. The invention comprises a system 10, which includes a fingertip sensor 12, which is in communication via a wire lead 14 with a device 16, which may be maintained in place on the arm of a user by a watchband 17 or the like. Device 16 is connected to fingertip sensor 12 by a lead 14, which can be positioned to extend from sensor 12 to device 16. Device 16 contains a miniature battery (not shown), a display 18, which may be readable more or less as a wristwatch is readable, and contacts 19 positioned in contact with the arm of the wearer to impart a small, but discernable electric shock, which may be of increasing severity to the user as the user's blood oxygen level declines further. This device also provides a visual readout 18 of the blood oxygen level and brings to the pilot's attention the information that the pilot's blood oxygen level is low by shocks from contacts 19. This device may also be used to sense the blood oxygen level for communication to a console on an instrument panel of an aircraft and it may also be used to activate other systems on the aircraft as will be discussed below. Further, display 18 may be resettable by the pilot by entering a code, by punching a button (not shown) a selected number of times or the like. Desirably the reset procedure will require sufficient pilot coordination to demonstrate that the pilot is functioning at a reasonable level of competence.

Fingertip sensors for sensing the blood oxygen level are available commercially and are available under the trademarks NONIN and NELLCOR from Nonin Medical, Inc., 2605 Fembrook Lane N., Plymouth, Minn. 55447 and Nellcor Puritan Bennett, Inc., 4280 Hacienda Drive, Pleasanton, Calif. 94588.

In FIG. 2, a variation of the present invention is shown. In this embodiment, an ear probe 22 is used in a helmet 20 to provide a measurement of the blood oxygen level of the pilot via the ear probe with the signal then being passed to a console display and control 24, which may be positioned on the instrument panel of the aircraft or at any other convenient spot. The console 24 includes a blood oxygen level indicator 26 where the blood oxygen level may be visually read. This readout also includes a reset 27, which allows the pilot to reset the indicator if necessary after an interval of low blood oxygen or the like. The console also contains an audible alarm activated by the oxygen blood level sensor. This alarm is shown as a shut-off switch 28 which can be used to shut-off the audible alarm. The console also includes a reset switch which desirably can be used to reset the blood oxygen level monitored for each of the controls or systems shown on the console. An autopilot switch 32, an auto-land switch 34, an altitude limiting system switch 36, an emergency oxygen supply switch 38, and pressurization switch 40 are shown. Each of these systems can be set for automatic activation in response to the signals from the ear probe 22, indicating a low pilot blood oxygen level or the systems can be activated by the pilot in response to a signal from the ear probe or the fingertip sensor.

It should be understood that each of these switches is shown as operable to disengage the respective system. Further each of the system is adapted to have the level of activation reset using reset switch 30. Each of these systems is readily activated in response to the low pilot blood oxygen sensor by any suitable program designed to activate one or more of the systems based upon the low blood oxygen level. This system can be powered by the electrical system of the aircraft and can be designed to transmit signals from the oxygen blood level sensor to the console electronically or via a wire lead connection or the like.

The degree of optimization of the software is completely optional with those skilled in the art. In other words, it may be desirable in some instances to activate only a portion of the systems typically present on a commercial aircraft or a larger private aircraft.

Desirably, the console also includes a master switch 42, which permits the pilot to shut down the console completely if it appears to be the proper corrective action at the time. In all instances, if capable of functioning, the pilot is clearly the best party to continue to pilot the aircraft. This system should be designed so that it may be reset or shut down only if the pilot is able to program in a simple code or otherwise demonstrate that he is capable of functioning. Otherwise, the systems may be activated to bring the aircraft to a safe altitude, safe pressure or cabin oxygen level or to safely land or fly the aircraft. The degree of sophistication included in the program is clearly a function of the needs of the particular aircraft involved.

In FIG. 1, a simple device is indicated that simply imparts a small and optionally an increasing level of shock to the wrist of the pilot should his blood oxygen level drop below a selected level. Desirably, device 16 also includes a shutoff allowing the pilot to disengage or reset the system by entering a simple code or the like. Any suitable exercise that demonstrates the pilot is in command of his senses and able to function normally is suitable to permit reactivation or shut off of the console.

It is well known that the normal blood oxygen levels of certain individuals vary. The blood oxygen saturation levels of smokers or older pilots consistently run lower than nonsmoking or younger pilots. Further, a high level of oxygen may need to be selected for night flying, where it has been shown that even slightly depressed oxygen saturation levels could adversely affect depth perception, color perception, etc.

The wrist-mounted display is battery operated and when the blood oxygen saturation level drops below the determined level, it sends an electric shock of about the same intensity as a pinch to the skin of the wrist. It does this repeatedly at certain preset intervals until it is reset by a pilot, who typically is required to key-in an appropriate code on the device to reset the device. If the device is not reset, the shock intensity should increase slightly periodically until the proper code is entered or the blood oxygen level has again risen to the desired levels.

The panel-mounted version is desirably used for higher altitude, higher performance aircraft. This unit would use a probe that fits in or on the pilot's earlobe with the probe being attached to a headset or helmet with wiring in electrical communication with an contact slot in the panel (console). The unit would display similar information as the portable device with audible alarms, possible activation of autopilot and/or auto-land, activation of emergency oxygen, activation of backup emergency pressurization, activation of rapid descent, etc., depending upon the configuration of the aircraft. It could also be programmed to alert air traffic control.

The console can also be programmed during start up procedures so that until the system is activated and set certain operations cannot take place, for instance, climbing above 15,000 feet, etc. The system can be made as simple or as sophisticated as needed to accommodate aircraft in commercial operations. Overriding the system can be accomplished by the pilot with specific codes and the like.

The system is not meant to restrict pilots but to assist them in recognizing hypoxia when it is still in an insidious state before it causes a serious problem. The system would be available in easy to use, easy to understand packages that are affordable and readily incorporated into existing aircraft.

As indicated previously, the fingertip blood oxygen level sensors are commercially available. Similarly, the earlobe probe sensors are also commercially available under the trademark NELLCOR from Nellcor Puritan Bennett, Inc.

The present invention provides both a method and an apparatus whereby the blood oxygen level of a pilot in an aircraft may be continuously or relatively continuously determined and a signal generated to increase aircraft safety by alerting the pilot to the onset of low blood oxygen levels of the pilot. The system may be of greater or lesser complexity than the basic system disclosed and may be on the order of the more complex system disclosed for commercial aircraft. The systems activated by the system and method of the present invention are available on commercial aircraft and could be readily activated as described herein by the use of a program responsive to the signals from the blood oxygen level sensor. Further, such systems are available for installation on aircraft if desired and may be installed to be activated in at least one mode by the blood oxygen level sensor.

While the present invention has been described by reference to certain of its preferred embodiments, it is pointed out that the embodiments described are illustrative rather than limiting in nature and that many variations and modifications are possible within the scope of the present invention. Many such variations and modifications may be considered obvious and desirable by those skilled in the art based upon a review of the foregoing description of preferred embodiments.

Having thus described the invention,

What is claimed is:

1. A system for increasing aircraft safety by monitoring and providing a signal indicative of the blood oxygen level of a pilot in an aircraft, the system comprising:
a) A blood oxygen level sensor in contact with a selected area of the pilot's body; and,
b) A signal generator in communication with the blood oxygen level sensor and adapted to generate a signal when the pilot's blood oxygen level drops below a selected blood oxygen level.

2. The system of claim 1 wherein the blood oxygen level sensor is a fingertip blood oxygen level sensor adapted to sense the pilot's blood oxygen level when the blood level sensor is positioned on one of the pilot's fingers.

3. The system of claim 2 wherein the blood oxygen level sensor is in communication with a wristband including the signal generator.

4. The system of claim 1 wherein the signal generator is adapted to administer a slight electrical shock signal to the pilot when the pilot's blood oxygen level drops below a selected minimum level.

5. The system of claim 1 wherein the blood oxygen level sensor is in communication with a visual signal generator that is indicative of the pilot's blood oxygen level.

6. The system of claim 1 wherein the blood oxygen level sensor is in communication with an audible signal generator adapted to generate an audible signal when the pilot's blood oxygen level drops below a selected minimum level.

7. The system of claim 1 wherein the blood oxygen level sensor is in communication with at least one of an autopilot system, an auto-land system, an altitude limitation system, an emergency oxygen supply system and a pressurization system.

8. The system of claim 1 wherein the pilot can reset the signal generator.

9. The system of claim 1 wherein the blood oxygen level sensor comprises an earlobe probe.

10. The system of claim 9 where in the earlobe probe is mounted in the pilot's helmet.

11. The system of claim 9 wherein the blood oxygen level sensor is in communication with at least one of a blood oxygen level visual indicator, an audible signal generator system, an autopilot system, an auto-land system, an emergency oxygen supply system, an altitude limiting system and a pressurization system.

12. The system of claim 11 wherein at least one of the systems is activated by a drop in the pilot's blood oxygen level below a selected minimum blood oxygen level.

13. The system of claim 11 wherein each system includes a pilot reset and override option.

14. A method for increasing aircraft safety by monitoring and providing a signal indicative of the blood oxygen level of a pilot in an aircraft, the method comprising:
a) monitoring the blood oxygen level of the pilot; and,
b) generating a signal indicative of the pilot's blood oxygen level when the pilot's blood oxygen level drops below a selected blood oxygen level.

15. The method of claim 14 wherein the pilot's blood oxygen level is monitored by a fingertip blood oxygen level sensor.

16. The method of claim 14 wherein the pilot's blood oxygen level is monitored by an earlobe probe.

17. The method of claim 14 wherein a signal indicative of a low blood oxygen level of the pilot is generated and communicated by at least one of a mild electric shock, a visual display and an audible signal to the pilot.

18. The method of claim 14 wherein the generation of a signal indicative of the pilot's low blood oxygen level may initiate at least one of a visual signal, an audible signal, a shock signal, an autopilot system procedure, an auto land system procedure, an emergency oxygen supply, an altitude limiting procedure and a pressurization procedure.

19. The method of claim 18 wherein the pilot may override the initiation of any of the signals and procedures.

20. The method of claim 14 wherein the selected minimum blood oxygen level is determined by a pre-flight test for the pilot.
